(19) United States
(12) Patent Application Publication
(10) Pub. No.: US 2024/0395363 A1
(43) Pub. Date: Nov. 28, 2024

(54) MULTI-REGION JOINT DETECTION

(71) Applicant: Illumina, Inc., San Diego, CA (US)

(72) Inventors: Shunhua Han, San Diego, CA (US); Daniel Andrews, Cambridge (GB); Vitor Ferreira Onuchic, San Diego, CA (US)

(21) Appl. No.: 18/674,279

(22) Filed: May 24, 2024

Related U.S. Application Data
(60) Provisional application No. 63/469,317, filed on May 26, 2023.

(51) Int. Cl. G16B 50/30; G16B 30/10; G16B 30/20; G16B 50/40; G16B 50/50

(52) U.S. Cl. CPC G16B 50/30; G16B 30/10; G16B 30/20; G16B 50/40; G16B 50/50

(57) ABSTRACT

Methods, systems, and apparatus, including computer programs encoded on computer-storage media, for multi-region joint detection. In some implementations, a method for identifying variants using multi-region joint detection in genetic sample sequences includes generating a set of candidate diplotypes mapped to at least two different regions in a reference sequence; generating a set of joint diplotype candidates comprising two or more candidate diplotypes of the set of candidate diplotypes; querying, for diplotypes at one or more locations of the at least two different regions in the reference sequence, a population database comprising genetic sequences of previously sequenced organisms; determining one or more values representing the frequency of specific diplotypes occurring within the genetic sequences of previously sequenced organisms; and generating, using the one or more values, an indication that the variant of the first joint diplotype candidate is an actual variant.

### CLAIM OF PRIORITY

This application claims priority under 35 USC § 119 (e) to U.S. Patent Application Ser. No. 63/469,317, filed on May 26, 2023, the entire contents of which are hereby incorporated by reference.

### FIELD

This specification generally relates to techniques for gene sequencing and comparison, e.g., of genomic data.

### BACKGROUND

Gene sequencing is a process that includes determining the order of nucleotides (A, C, G, and T) in a deoxyribonucleic acid (DNA) molecule. Instances of the nucleotide adenine in genomic data can be represented in a sequence by the letter “A.” Similarly, instances of nucleotides guanine, cytosine, thymine, or uracil in ribonucleic acid (RNA), can be represented by “G”, “C”, “T”, or “U”, respectively.

Genomic sequencing can be combined with genomic read mapping to identify the locus of a gene and the distances between genes. Computers can be used to analyze one or more sets of genomic data and correlate a collection of molecular markers, such as a series of nucleotides, with their respective positions on a given reference genome. In this way, a computer can be used to “map” the collection of molecular markers onto the reference genome.

### SUMMARY

Techniques described in this document include identifying genetic variants, e.g., using joint diplotype candidates. A variant can include any base call (e.g., A, C, G, and T) in a sample sequence being different from a reference sequence at a given location. For example, typical human DNA can include upwards of 3 billion bases. The sequence of the bases can provide an indication of certain entity characteristics such as medical abnormalities, such as certain types of cancer, heart disease, among others, as well as characteristics, such as hair or eye color. Thus, identifying variants that indicate how a biological entity's sample sequence differs from a known reference sequence can be especially important in predictive medical care or treatment.

In order to detect variants, a given sample sequence needs to be matched to a given reference location. Detecting variants for regions with sequences that are common can be difficult because the mapping location can be ambiguous. The proposed techniques include identifying multiple regions of sample sequences which have ambiguous mapping locations and creating candidate groups iteratively—where each candidate group includes a potential mapping of a given sample sequence (e.g., a haplotype) to a reference sequence. Some candidate mappings may support a variant while some may support non-variants or a homozygous pairing of alleles.

The techniques described in this document improve such multi-region joint detection by using population databases to help determine the probability of a given variant. The probability can depend on a location within a genomic sequence. For example, the probability of a particular variant at a first position of a gene may be high relative to the probability of the same variant at the second position of the gene. Systems described in this document can calculate multiple probabilities for different types of variants to determine what combination of sample sequence mappings to reference sequence is most likely and, therefore, what areas of the sample sequence likely include variants.

The techniques described can include adjusting population-based probabilities using specific sequence data of a sample. In some implementations, a processing system determines a similarity between a sample and one or more historically obtained genetic sequences. For example, the sample may have originated from a similar species or a same species living in a similar area or having similar characteristics. By adjusting the probabilities based on the sample sequence, systems described in this document can account for the fact that genetically similar organisms can have similar types of variants at particular locations along a genetic sequence (e.g., at specific locations along one or more genes).

Advantageous implementations can include one or more of the following features. For example, accuracy of variant detection can be improved by accounting for either, or both, previously identified variants in population databases or previously identified variants in populations that are within a threshold similarity compared to an organism providing a sample being tested. Techniques described can help solve the longstanding problem of accurately detecting variants within paralogous or homologous regions of DNA. These regions, because of how often they appear in a genetic sequence, can lead to incorrect mapping and subsequent incorrect variant detection. Using a joint detection approach with population-based data, techniques described herein can accurately detect variants in these difficult regions. Techniques described in this document can be applied to other forms of genetic sequences, such as ribonucleic acid (RNA).

Significantly, not only does the present disclosure provide the improvements described above and solve the aforementioned longstanding problems in the art, but the present disclosure achieves these benefits in a manner that provides significant increases in performance. Specifically, as described herein with reference to FIGS. 6A and 6B, the present disclosure provides these benefits while also achieving increase in variant recall and precision over prior implementations.

One innovative aspect of the subject matter described in this specification is embodied in a method that includes obtaining a plurality of haplotypes from one or more reads of a biological sample; generating a set of candidate diplotypes mapped to at least two different regions in a reference sequence; generating a set of joint diplotype candidates comprising two or more candidate diplotypes of the set of candidate diplotypes, wherein the set of joint diplotype candidates includes a first joint diplotype candidate indicating a variant in at least one base from the reference sequence; querying, for diplotypes at one or more locations of the at least two different regions in the reference sequence, a population database comprising genetic sequences of previously sequenced organisms; determining one or more values representing the frequency of specific diplotypes occurring within the genetic sequences of previously sequenced organisms; and generating, using the one or more values representing the frequency of specific diplotypes occurring within the genetic sequences of previously sequenced organisms, an indication that the variant of the first joint diplotype candidate is an actual variant.

Other implementations of this and other aspects include corresponding systems, apparatus, and computer programs, configured to perform the actions of the methods, encoded on computer storage devices. A system of one or more computers can be so configured by virtue of software, firmware, hardware, or a combination of them installed on the system that in operation cause the system to perform the actions. One or more computer programs can be so configured by virtue of having instructions that, when executed by data processing apparatus, cause the apparatus to perform the actions.

The foregoing and other embodiments can each optionally include one or more of the following features, alone or in combination. For instance, in some implementations, actions include identifying the at least two different regions in the reference sequence as paralogous or homologous regions.

In some implementations, generating the set of joint diplotype candidates includes: generating permutations of haplotypes, from among the plurality of haplotypes, and mapping regions, from among the at least two different regions in the reference sequence, for all haplotypes of the plurality of haplotypes and all regions of the at least two different regions in the reference sequence.

In some implementations, determining the one or more values representing the frequency of specific diplotypes occurring within the genetic sequences of previously sequenced organisms includes: determining a quantity for each unique diplotype in the genetic sequences of previously sequenced organisms, wherein the quantity represents a number of distinct genetic sequences with the given unique diplotype at a given location; and determining a quantity of the genetic sequences of previously sequenced organisms.

In some implementations, determining the one or more values representing the frequency of specific diplotypes occurring within the genetic sequences of previously sequenced organisms includes: determining a quantity for each unique diplotype in the genetic sequences of previously sequenced organisms, wherein the quantity represents a number of distinct genetic sequences with the given unique diplotype at a given location; determining a quantity of the genetic sequences of previously sequenced organisms; determining one or more similarity values representing a similarity between a portion of the one or more reads of the biological sample and portions of the genetic sequences of previously sequenced organisms at one or more positions not included in the at least two different regions in the reference sequence; and adjusting, using the one or more similarity values, values representing the quantity for each unique diplotype relative to the quantity of the genetic sequences; and determining the adjusted values as the one or more values representing the frequency of specific diplotypes occurring within the genetic sequences of previously sequenced organisms.

In some implementations, determining the one or more similarity values includes: determining a number of variants between the portion of the one or more reads of the biological sample and the portions of the genetic sequences of previously sequenced organisms at the one or more positions not included in the at least two different regions in the reference sequence.

In some implementations, actions include determining one or more values representing a proximity of the number of variants to one of the at least two different regions in the reference sequence. In some implementations, the proximity represents a number of base pairs from where a variant of the number of variants occurs to a starting position of the one of the at least two different regions in the reference sequence.

In some implementations, generating, using the one or more values representing the frequency of specific diplotypes occurring within the genetic sequences of previously sequenced organisms, an indication that the variant of the first joint diplotype candidate is an actual variant includes: generating, using the one or more values representing the frequency of specific diplotypes occurring within the genetic sequences of previously sequenced organisms, one or more values representing an a-priori probability; generating, using the generated one or more values representing the a-priori probability, one or more values representing an a-posteriori probability

In some implementations, generating the one or more values representing the a-priori probability includes: combining, based on a location and composition of each diplotype in the first joint diplotype candidate, two or more of the one or more values representing the frequency of specific diplotypes occurring within the genetic sequences of previously sequenced organisms.

In some implementations, combining the two or more of the one or more values representing the frequency of specific diplotypes occurring within the genetic sequences of previously sequenced organisms includes: multiplying the two or more of the one or more values.

In some implementations, the one or more values representing the frequency of specific diplotypes occurring within the genetic sequences of previously sequenced organisms comprises 16 values. In some implementations, the 16 values are between 0 and 1.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 is a diagram showing an example of a system for multi-region joint detection that detects variants using a custom prior model generated based on a population database.

FIG. 2 is a diagram showing an example of a system for multi-region joint detection using population-based prior probabilities.

FIG. 3 is a diagram showing an example of a system for multi-region joint detection using sample specific population-based prior probabilities.

FIG. 4 is a flow diagram illustrating an example of a process for multi-region joint detection.

FIG. 5 is a diagram illustrating an example of a computing system used for multi-region joint detection.

FIG. 6A and FIG. 6B are diagrams illustrating performance of the described techniques.

Like reference numbers and designations in the various drawings indicate like elements.

### DETAILED DESCRIPTION

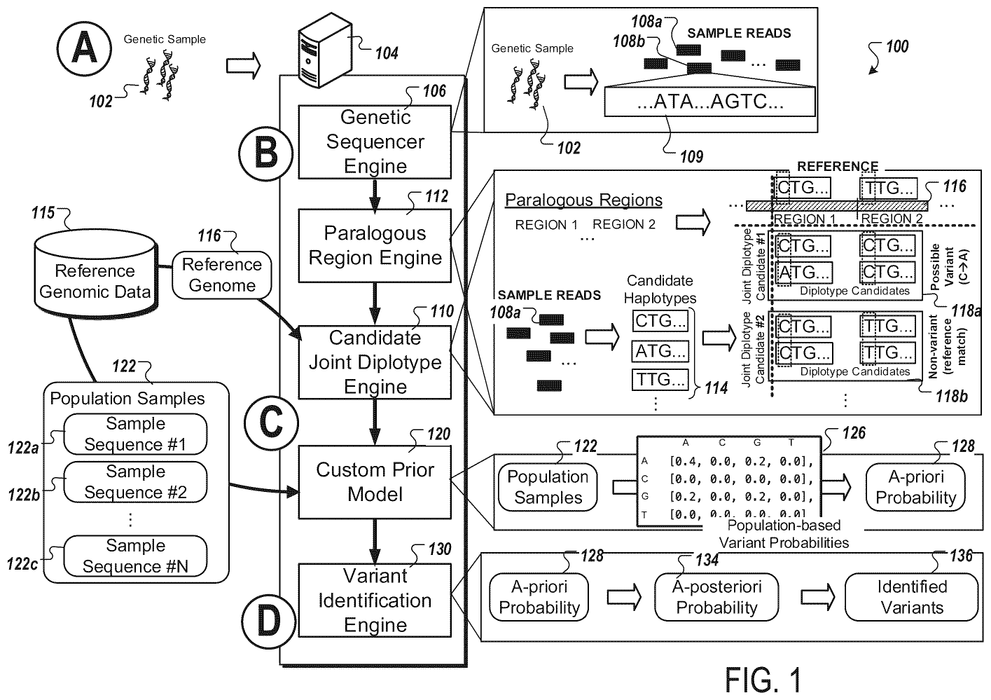

FIG. 1 is a diagram showing an example of a system 100 for multi-region joint detection that detects variants using a custom prior model generated based on a population database. The system 100 includes a control unit 104 and database 115. The control unit 104 can include, or be communicably connected to, one or more processing devices to perform operations described in reference to the control unit 104. The control unit 104 can access data stored on the database 115, such as population samples 122.

In general, FIG. 1 shows candidate variants being identified and processed to determine if any of the candidate variants are actual variants. FIG. 1 is described with reference to stages A through D. Although described in order from stage A through D, operations of the system 100 can occur in different orders or in parallel. For example, the system 100 can engage in one or more parallel processing operations, such as identifying paralogous regions and generating a custom prior model. In some implementations, the control unit 104 previously generates one or more custom models before identifying one or more paralogous regions or sample sequences for processing. The control unit 104 can then query a database to obtain one or more custom models for processing.

In stage A, the control unit 104 obtains genetic sample data. The genetic sample data can be a genetic sample from an organism, such as a dog, human, or the like. The genetic sample can indicate DNA or RNA of the organism. The control unit 104 can be used to identify genetic variants with confidence values in the given organism based on detected variants in the genetic sequence.

In stage B, the control unit 104 processes the genetic sample 102. For example, a genetic sequencer engine 106 of the control unit 104 can sequence the genetic sample 102 to generate a sequence list of “base calls” or “bases” present in the DNA of the genetic sample 102. Sample reads 108a-b are shown in FIG. 1. In general, the genetic sequencer engine 106 can generate hundreds or thousands of sequences. An example sequence stretch of sample read 108b is shown in item 109 as “ . . . ATA . . . AGTC . . . ” representing the identification, by the genetic sequencer engine 106, of a sequence of adenine (A) followed by thymine (T) followed by adenine (A) and adenine (A) followed by guanine (G) followed by thymine followed by cytosine (C).

The genetic sequencer engine 106 can be included in the control unit 104 or be communicably connected to the control unit 104. In some implementations, the control unit 104 includes one or more processors that provide or obtain data from a genetic sequencer machine configured to perform next-generation “short-read”, third-generation “long-read” sequencing methods, among others. In some implementations, the genetic sequencer engine 104 can include a next generation nucleic acid sequencer that, in addition to performing next-generation nucleic acid sequencing, also includes computing resources necessary to perform the operations of the genetic sequencer engine 106, paralogous region engine 112, candidate joint diplotype engine 110, custom prior model 120, and the variant identification engine 130.

The control unit 104 identifies one or more paralogous regions. For example, the control unit 104 can operate a paralogous region engine 112 that identifies one or more paralogous regions. In some implementations, the control unit 104 identifies paralogous regions in a reference genome, such as a reference genome 116 obtained from the database 115. For example, the control unit 104 can compare one or more sequences of the reference genome and determine sequences that satisfy one or more similarity thresholds as paralogous or homologous regions. In some implementations, the database 115 includes one or more processors communicably connected to one or more electronic storage devices configured to store digital data. The database 115 can store digital data representing the population samples 122 and the reference genome 116.

In some implementations, the paralogous region engine 112 obtains input from a user. For example, the paralogous region engine 112 can obtain input from a user indicating specific paralogous or homologous regions. Genes where the paralogous region engine 112 identifies paralogous or homologous regions can include, for example, PMS2 or PMS2CL among other genes.

In some implementations, the control unit 104 identifies paralogous regions using base sequences generated from the genetic sample 102. For example, the paralogous region engine 112 can compare one or more regions from one or more sequenced portions of the genetic sample 102. The paralogous region engine 112 can identify regions that satisfy one or more similarity thresholds.

In general, determining whether or not one or more regions, either in the sample or reference sequence, satisfy one or more similarity thresholds can include one or more of determining one or more same bases are in the same location in multiple regions, above a percentage number of same bases are in the same location, among other methods.

In some implementations, the candidate joint diplotype engine 110 obtains a reference genome 116. For example, the candidate joint diplotype engine 110 can obtain the reference genome 116 from the database 115 for reference genomic data. The reference genome 116 can indicate a known full or partial sequence of entity or class of entities, where an entity can include any plant, animal, organism, or a class thereof. The reference genome 116 can include reference bases in multiple positions within multiple genes. Deviation from the reference genome 116 at any given position can be considered a variant. The notion of a variant can be described with reference to the following example. First, assume, for example, a sample sequence from the genetic sample 102 matches the reference 116 in positions 1-5 along the genetic sequence of the reference 116. Next, however, in position 6 of the same example, the reference 116 can indicate adenine where the sample sequence indicates thymine or some other nucleotide. If the sample is correctly mapped to this portion of the reference, e.g., based on positions 1-5 matching the reference, the sample sequence has a variant at the position 6.

Because the identified homologous or paralogous regions are identical or highly similar (e.g., differing in only one or two bases for a given sequence length), mapping, especially short read sequences, to these regions can be difficult. The control unit 104 helps to solve this problem by generating joint diplotype candidates from haplotypes that have been identified as potentially being mapped to one or more of the homologous or paralogous regions. Each joint diplotype candidate can include at least one candidate diplotype. Each candidate diplotype can include two or more haplotypes combined to form the candidate diplotype.

In the example of FIG. 1, the candidate joint diplotype engine 110 generates joint diplotype candidates #1 118a and #2 118b, among others. Joint diplotype candidates #1 118a includes two candidate diplotypes. The first is a pairing of haplotype “CTG . . . ” with haplotype “ATG . . . ” and the second is a pairing of haplotype “CTG . . . ” and haplotype “CTG . . . ” which can be the same or different. The first candidate diplotype of joint diplotype candidates #1 118a is mapped to REGION 1 and the second candidate diplotype of joint diplotype candidate #2 118a is mapped to REGION 2.

As shown in FIG. 1, joint diplotype candidates #2 118b similarly includes two candidate diplotypes. The first of these is a pairing of haplotype “CTG . . . ” with haplotype “CTG . . . ” and the second is a pairing of haplotype “TTG . . . ” and haplotype “TTG . . . ” which can be the same or different. Similar to candidate #1 118a, the first candidate diplotype of joint diplotype candidate #2 118b is mapped to REGION 1 and the second candidate diplotype of joint diplotype candidate #2 118a is mapped to REGION 2.

Joint diplotype candidate #1 118a supports a potential variant in the first position at REGION 1 because the first candidate diplotype at the first position includes C and A where the reference is C. Because the first candidate diplotype includes an A instead of C, the first candidate diplotype can be a potential variant. Joint diplotype candidate #1 118a supports another potential variant in the first position at REGION 2 because the second candidate diplotype at the first position of REGION 2 includes C and C where the reference is T. Because the second candidate diplotype includes C and C instead of T, the second candidate diplotype can be a potential variant. Joint diplotype candidate #2 118b is a non-variant candidate where no positions support a variant.

In general, the candidate joint diplotype engine 110 can generate joint diplotype candidates for as many regions that are identified as homologous or paralogous. In some implementations, the candidate joint diplotype engine 110 generates joint diplotype candidates that include hundreds or thousands of candidate diplotypes mapped to the hundreds or thousands of paralogous or homologous regions. In some implementations, the candidate joint diplotype engine 110 randomly permutes the identified candidate haplotypes for all identified regions until all permutations are generated. The set of all joint diplotype candidates can include a full set of all possible permutations of the identified candidate haplotypes mapped to the identified regions.

The question can be presented, which candidates correspond to actual mappings to the reference genome 116? It is possible that either joint diplotype candidate #1 118a or joint diplotype candidate #2 118b is the correct mapping. Each mapping would lead to different determinations regarding variants. For example, if joint diplotype candidate #1 118a is the correct mapping, the genetic sample 102 displays at least 2 variants from the reference. On the other hand, if joint diplotype candidate #2 118b is the correct mapping, the genetic sample 102 does not display variants. A determination one way or another can have extreme side effects when used as the basis for treatment or medical predictions. Some treatments for organisms with one variant could be deadly or otherwise harmful to other organisms without the variant. Similarly, diseases common based on one or more variants may be uncommon or non-existent without the one or more variants.

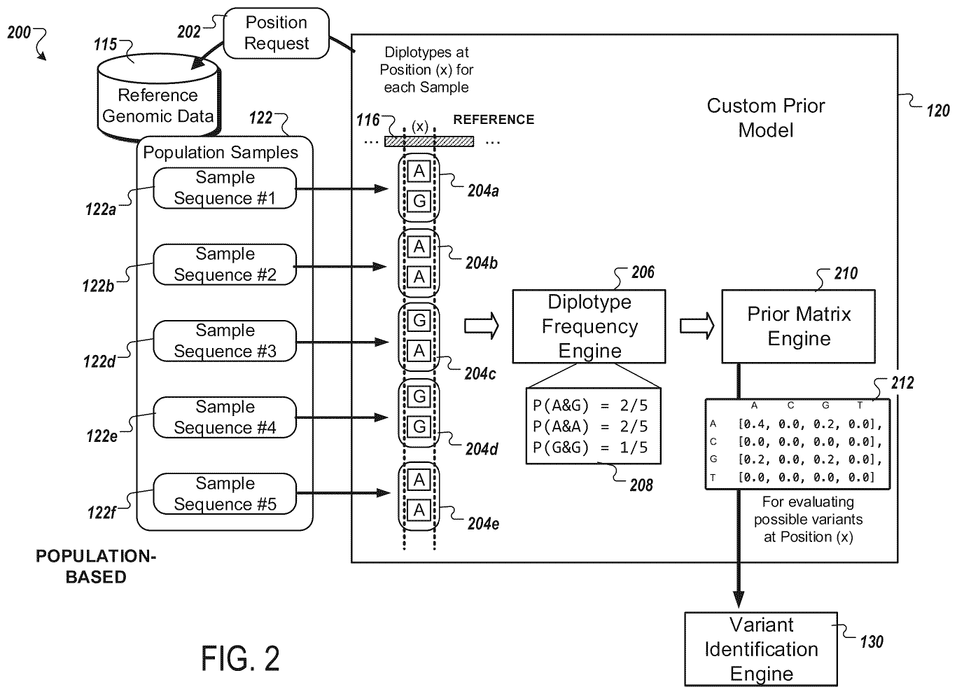

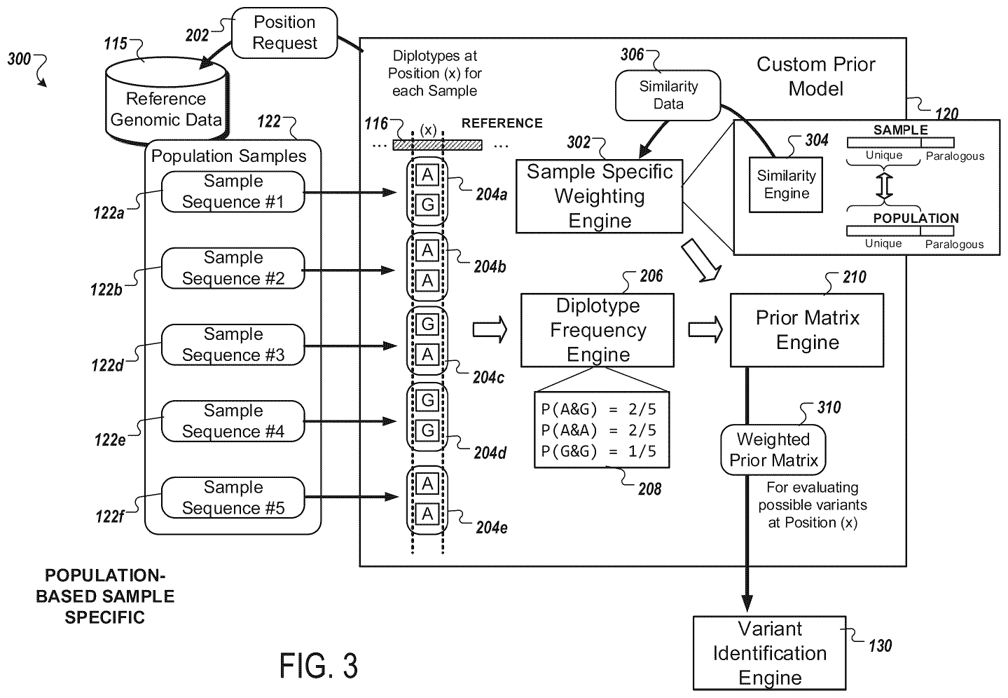

In stage C, the control unit 104 generates prior probability values to help determine which of the joint diplotype candidates are likely to be correct. Specific implementations are discussed in FIG. 2 and FIG. 3. In general, a custom prior model 120 of the control unit 104 can obtain population samples 122. The population samples 122 can include sample sequences 122a-c. Each sample sequence can indicate previously sequenced genetic material from a same or different organism. The custom prior model 120 can generate a set of probability prior values 126 and generate, using the set of probability prior values 126, an a-priori probability 128.

In stage D, the control unit 104 determines whether the genetic sequence of the genetic sample 102 supports one or more variants. For example, a variant identification engine 130 of the control unit 104 can use the a-priori probability 128 generated based on the custom prior model 120 to generate an a-posteriori probability 134. The control unit 104 can use the a-posteriori probability 134 to generate one or more values indicating the likelihood of one or more of the generated joint diplotype candidates being correct and the associated variants being true variants. The variant identification engine 130 can generate data indicating identified variants 136. The identified variants 136 can include variants in genes, such as PMS2 and PMS2CL among others.

In some implementations, the variant identification engine 130 determines that one or more values indicating a likelihood of the joint diplotype candidate #1 118a being correct and the associated variants being true variants satisfies one or more thresholds. For example, the variant identification engine 130 can determine that one or more values representing the joint diplotype candidate #1 118a are greater than or equal to one or more other values representing other generated joint diplotype candidates. In some implementations, the variant identification engine 130 ranks one or more values to determine a candidate with a highest value as the correct candidate. Data of the identified variants 136 can indicate a position or gene and what variant is present there.

FIG. 2 is a diagram showing an example of a system 200 for multi-region joint detection using population-based prior probabilities. FIG. 2 expands on possible implementations of the custom prior model 120 and how it generates prior probability values for use by the variant identification engine 130. By using population data, the system 200 can be used with the system 100 to increase the accuracy of identifying variants given a frequency of such variants occurring in previously sequenced genetic data.

The example of FIG. 2 shows the custom prior model 120 generating and transmitting a position request 202 to the database 115. In response, the custom prior model 120, or implementing one or more processors, such as the control unit 104, can obtain the population samples 122. In some implementations, the custom prior model 120 previously stores population samples and queries for the previously stored samples in connected databases. In some implementations, the position request 202 indicates a specific position. For example, the custom prior model 120 can generate a request for genetic data in a population for a particular reference position (x) where (x) can be a position within a gene in a genetic sequence. In some implementations, the position request 202 does not include a specific position. For example, the custom prior model 120 can request population sample data (e.g., before or after the control unit 104 identifies candidate locations or obtains sequenced sample data) and obtain the population sample data including one or more genetic sequences. For a given position, the custom prior model 120 can query the obtained sample data for a particular position.

The custom prior model 120 obtains data for the population samples 122. For example, the custom prior model 120 can obtain data indicating genetic sequences 122a-b and 122d-f. The genetic sequences 122a-b and 122d-f can include genetic data for a particular position (x) along the reference genome 116. For example, the genetic sample #1 122a can indicate that a corresponding organism from which the genetic sample #1 122a was generated had the base pair A and G, diplotype 204a, at the position (x). Similarly, genetic sample #2 122b indicates diplotype A and A 204b at position (x), genetic sample #3 122d indicates diplotype G and A 204c at position (x), genetic sample #4 122e indicates diplotype G and G 204d at position (x), and genetic sample #5 122f indicates diplotype A and A 204e at position (x).

In some implementations, variants include single-nucleotide polymorphisms (SNPs) and small insertions/deletions (INDELs). The techniques described in this document can be used for SNP variant identification and INDEL variant identification, among other variant types. For example, the custom prior model 120 can be used to generate a-priori probabilities (e.g., a-priori probability 128) using SNP or INDEL data. INDEL data can include two or more nucleotides from one or more organisms in the reference genomic data 115. SNP data can include single nucleotide sequence suitable for SNP variant detection. The SNP implementation version is described in detail in this document. Similar techniques can be used to detect variants in two or more nucleotides, e.g., INDELs.

In some implementations, the position (x) shown in FIG. 2 includes a set of two or more positions. For example, for identifying INDEL variants, population data can be obtained, e.g., by the custom prior model 120. The population data can include nucleotide sequence data for multiple positions. FIG. 2 shows an implementation where the custom prior model 120 generates a-priori probabilities using single positions—e.g., a single pair of nucleotides for a given known position along a reference. For detecting INDEL variants, the custom prior model 120 can obtain multiple positions, including multiple pairs of nucleotides or haplotypes, from the reference genomic data 115. The multiple positions can be provided by the custom prior model 120 to the diplotype frequency engine 206. In some implementations, the diplotype frequency engine 206 counts a number of unique pairings of nucleotides over two or more reference positions (e.g., how many instances of A and A paired with A and G and how many instances of A and A paired with A and T, among others). In some implementations, the custom prior model 120 generates a matrix of values for generating a-priori probabilities using a pair of haplotypes obtained from the reference genomic data 115 corresponding to a given region in the reference.

The custom prior model 120 provides data of the genetic sequences 122a-b and 122d-f to a diplotype frequency engine 206. The diplotype frequency engine 206 determines a frequency of each diplotype in the population obtained from the database 115. The diplotype frequency engine 206 can include one or more processors of the custom prior model 120 or the control unit 104.

Frequency counts generated by the diplotype frequency engine 206 are shown in item 208. Of course, 5 samples are shown for illustration simplicity but the population database can include many more, e.g., hundreds or millions. The diplotype frequency engine 206 determines that ⅖ths of the population have the diplotype A and G at the position (x), ⅖ths of the population have the diplotype A and A at the position (x), and ⅕th of the population have the diplotype G and G at the position (x).

The position (x) can correspond to any position of the reference genome 116. For example, the position (x) can include one or more of the positions of REGION 1 and REGION 2 as shown in FIG. 1. The custom prior model 120 can determine a frequency count in a given population for diplotypes at each location along a reference being matched.

The custom prior model 120 provides data from the diplotype frequency engine 206 to a prior matrix engine 210. The prior matrix engine 210 can use the data indicating the frequency of one or more diplotypes in a population to generate values of a matrix and provide the matrix, or associated data such as a-priori probability values, to the variant identification engine 130.

As shown in FIG. 2, the generated matrix 212 includes 0.4 in the portion of the matrix indicating a probability of A and A, 0.2 and 0.2 in the portions of the matrix indicating a probability of A and G or G and A, and 0.2 in the portion of the matrix indicating a probability of G and G. These values correspond to the frequencies with which the corresponding diplotypes have been identified to occur in the population samples 122.

To generate an a-priori probability, the control unit 104 can combine probabilities for all positions of a joint diplotype candidate. For example, for the joint diplotype candidate #1 118a, the custom prior model 120 can generate specific matrices for each position corresponding to the matched REGION 1 and REGION 2. The values of each matrix can be used by the custom prior model 120 to generate a probability value for each position of the joint diplotype candidate #1 118a. For example, if position (x) corresponds to position 1 of REGION 1, the custom prior model 120 can identify a probability of CA, in the first position of the first candidate diplotype of the joint diplotype candidate #1 118a, using the matrix 212. The custom prior model 120 can identify the probability as 0. However, in a real case, zero values in the matrix may be unlikely but can be dependent on a size of samples being used to generate the population-based probabilities.

In some implementations, the custom prior model 120 generates one or more matrices for reference positions before processing one or more sample reads. In this way, the custom prior model 120 can efficiently query matrix values without generating new matrix values for each position along a reference.

The probabilities can be combined to generate an a-priority probability, such as the a-priori probability 128 of FIG. 1. Combination can be performed by the custom prior model 120 or other element of the control unit 104. The custom prior model 120 can be used to identify the probability of each diplotype occurring at the corresponding reference location for an entire joint diplotype candidate. This can include querying multiple matrices corresponding to population-based probabilities of diplotypes occurring at locations along the reference. The custom prior model 120 can generate an a-prior probability for each joint diplotype candidate as a multiplicative product or log sum of each individual diplotype probability.

In some implementations, the control unit 104 operates one or more processes to generate output of a Bayesian theorem. In some implementations, the control unit 104 computes one or more expressions, such as P(Gₘ|R)=(P(R|Gₘ) P (Gₘ))/(Σᵢ₌₁^{M}P(R|Gᵢ)P(Gᵢ)) where P(Gₘ) can represent the combination of probabilities from all paralogous or homologous regions (e.g., REGION 1 and REGION 2 of FIG. 1 or multiple other regions in more complex scenarios). The one or more expressions can be used to determine, based on population probabilities, the likelihood that a given variant is an actual variant.

N can represent the number of regions to be jointly processed (e.g., REGION 1 and REGION 2), Hₖ can represent candidate haplotypes (e.g., candidate haplotypes 114) where k=1 . . . K and each can include various SNPs, insertions and/or deletions relative to a reference sequence (e.g., the reference genome 116), Gₘ can represent candidate solutions for both phases ϕ=1,2 and all regions n=1 . . . N (e.g., joint diplotype candidate #1 118a), G_{mϕn} can be a candidate solution generated from the set of candidate haplotypes {H₁ . . . Hₖ}, and r; can represent paired reads {r_{i,1}, r_{i,2}}. The probability of each candidate haplotype can be expressed as P (r; (Hₖ)=P(r_{i,1}|Hₖ)P(r_{i,2}|Hₖ). The conditional probability of each read for each candidate solution Gₘ can be expressed as

$$P ( {r}_{i} \vert {G}_{m} ) = \frac{1}{2 N} {\sum }_{n = 1}^{N} {\sum }_{ϕ = 1}^{2} ( {r}_{i} \vert {G}_{m , ϕ , n} )$$

and a conditional probability of an entire pileup R={r₁ . . . r_{N}_{_{R}}} can be expressed as P(R|Gₘ)=Πᵢ₌₁^{N}^{_{R}} P(r₁(Gₘ). The a-posteriori probability, such as the a-posteriori probability 134 of FIG. 1, can be expressed as P(Gₘ|R)=(P(R|Gₘ)P(Gₘ))/(Σᵢ₌₁^{M}P(R|Gᵢ)P(Gᵢ)). The P(Gₘ) included in the expression for a-posteriori probability can be represented in item 128 of FIG. 1 and generated by the system 100 using the probability values generated by the system 200.

The relative probability of each candidate can be expressed as

$$\frac{P ( {v}_{j} \vert R )}{P ( ref \vert R )} = \frac{{Σ}_{( m \vert {G}_{m} \to {v}_{j} )} P ( {G}_{m} \vert R )}{{Σ}_{( m \vert {G}_{m} \to ref )} P ( {G}_{m} \vert R )}$$

where Gₘ→vⱼ indicates that Gₘ supports variant vⱼ and Gₘ→ref indicates that Gₘ supports the reference. A corresponding quality score can be reported as

$$QUAL ( {v}_{j} ) = - 10 log\_ 10 ( \frac{P ( {v}_{j} \vert R )}{P ( ref \vert R )} ) ,$$

e.g., in a variant call file (VCF) or using other file formats.

In some implementations, the control unit 104 generates one or more outputs using the expressions referenced in this document to identify whether or not a variant is an actual variant (e.g., using the variant identification engine 130).

FIG. 3 is a diagram showing an example of a system 300 for multi-region joint detection using sample specific population-based prior probabilities. Similar to the system 200 of FIG. 2, the system 300 of FIG. 3 obtains population data from the database 115. A difference is that the system 300 is sample specific. The system 300 includes the sample specific weighting engine 302. The sample specific weighting engine 302 determines a weighting value for one or more determined frequencies by comparing sequences of a sample to sequences of a population sequence. In general, probabilities of population samples are increased when the population sample is more similar to the sample being tested (e.g., the genetic sample 102) compared to another population sample. For example, the sample specific weighting engine 302 can determine that the sample sequence 122a is more similar to a sample than the sample sequence 122b. The sample specific weighting engine 302 can determine a similarity using a similarity engine 304.

In some implementations, the similarity engine 304 identifies one or more bases in a region that is not identified (e.g., by the paralogous region engine 112) as paralogous or homologous. The similarity engine 304 can compare one or more bases of the region that is not identified as paralogous or homologous with corresponding portions of the sample sequences from the population (e.g., sample sequence #1 122a, 122b, among others). In some implementations, the similarity engine 304 identifies the number of variants in the region that is not identified as paralogous or homologous. The number of variants can be inversely proportional to a subsequent weighting applied to the probability associated with the diplotype expressed by the given historical sample.

For example, the similarity engine 304 can identify 40 differences between the sample sequence #1 122a and a given sample being tested (e.g., sequenced data corresponding to the genetic sample 102) in the region that is not identified as paralogous or homologous. The similarity engine 304 may also identify 20 differences between the sample sequence #2 122b and the same given sample being tested (e.g., sequenced data corresponding to the genetic sample 102) in the same region that is not identified as paralogous or homologous. The similarity engine 304 can provide similarity data 306 to the sample specific weighting engine 302 that indicates the various similarities between a given sample and one or more population sequences (e.g., one or more values indicating a number of variants present in regions not identified as paralogous or homologous). In the above example, the sample specific weighting engine 302 can generate a weight for the diplotype of the sample sequence #2 122b that increases that probability compared to the diplotype of the sample sequence #1 122a, e.g., for position (x) in the reference 116 or other positions along the reference 116. In some implementations, the weights are generated for each region. In some implementations, weightings are generated for each position in each region.

The sample specific weighting engine 302 provides data to the prior matrix engine 210. The prior matrix engine 210 can adjust the frequency data provided by the diplotype frequency engine 206 using the weightings provided by the sample specific weighting engine 302 to generate a weighted prior matrix 310. In general, the weighted prior matrix 310 can increase probabilities of diplotypes that appear in organisms similar to the organism from which the sample being tested originated. The similarity can be determined by the similarity engine 304 as described in this document.

In some implementations, the similarity data 306 indicates a proximity to a homologous or paralogous region. For example, the similarity data 306 can indicate variants between a given sample a sample sequence from the database 115 at a location that is not identified as homologous or paralogous (e.g., to the right or left in the genetic sequence of reference 116 from REGION 1 or REGION 2). However, the similarity engine 304 can also indicate in the similarity data 306 a proximity of such variants to the identified homologous or paralogous regions. Because the goal can be described as finding or imputing the likely variants in a given section of a sample genetic sequence compared to a reference, the system 300 using the sample specific weighting engine 302 can increase accuracy by weighting probabilities more for population samples that have fewer variants close to the identified homologous or paralogous regions, e.g., in the sample 102.

In some implementations, the sample specific weighting engine 302 obtain information from the similarity engine 304 indicating variants and location of the variants. As described, the sample specific weighting engine 302 can generate weightings that effectively increase the a-priori probability of diplotypes that appear in organisms genetically similar to a sample organism being tested. Genetically similar organisms can include one or more of variant counts in base call sequences around regions of interest—e.g., paralogous or homologous regions—or the proximity of such variants relative to the regions of interest.

The weighted matrix 310 can be used, e.g., by the control unit 104, similar to the matrix 212. The form of the weighted matrix 310 can also be similar (e.g., 4 by 4 values) where the values are population-based frequencies scaled by weightings determined by the sample specific weighting engine 302. The weighted matrix 310 can be used, e.g., by the custom prior model 120 to generate a-priori probability 128 for one or more joint diplotype candidates, otherwise referred to as P(Gₘ). The control unit 104 can use the generated a-priori probabilities 128 to identify whether or not a variant is an actual variant, e.g., by calculating the a-posteriori probability 134 using expression P(Gₘ|R)=(P(R|Gmm)P(Gₘ))/(Σᵢ₌₁^{M}P(R|Gᵢ)P(Gᵢ)) described in this document.

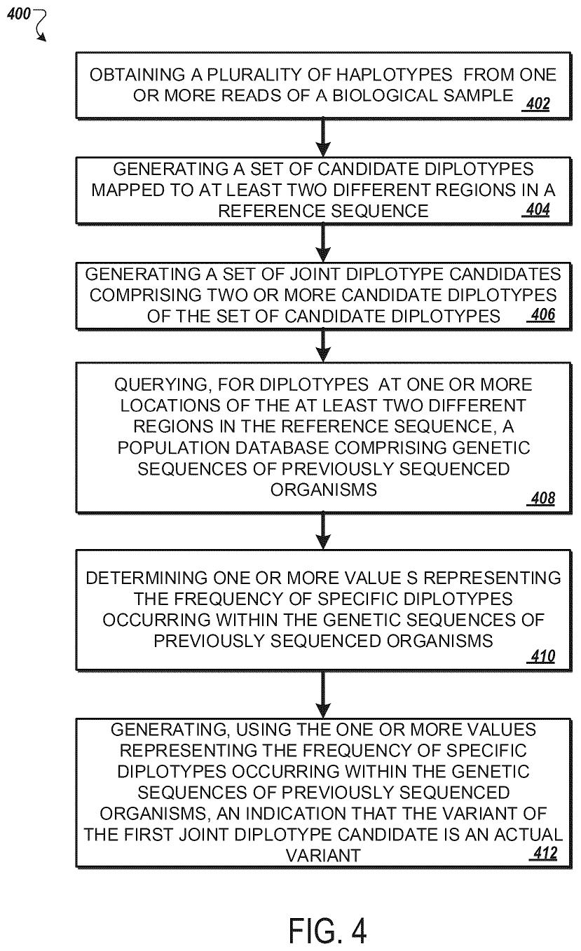

FIG. 4 is a flow diagram illustrating an example of a process 400 for multi-region joint detection. The process 400 may be performed by one or more electronic systems, for example, the system 100 of FIG. 1, the system 200 of FIG. 2, or the system 300 of FIG. 3.

The process 400 includes obtaining a plurality of haplotypes from one or more reads of a biological sample (402). For example, the control unit 104 can obtain, from the one or more sample reads 108a-b, one or more candidate haplotypes 114.

The process 400 includes generating a set of candidate diplotypes mapped to at least two different regions in a reference sequence (404). For example, the control unit 104 can generate diplotype candidates (e.g., including C and A in REGION 1 and C and C in REGION 2, among other nucleotides).

The process 400 includes generating a set of joint diplotype candidates comprising two or more candidate diplotypes of the set of candidate diplotypes (406). In some implementations, the set of joint diplotype candidates includes a first joint diplotype candidate indicating a variant in at least one base from the reference sequence. The control unit 104 can generate joint diplotype candidates #1 118a and #2 118b, among others. The control unit 104 can generate one or more joint diplotype candidates from generated diplotype candidates. The joint diplotype candidates can be generated by permutating a set of two or more diplotype candidates.

The process 400 includes querying, for diplotypes at one or more locations of the at least two different regions in the reference sequence, a population database comprising genetic sequences of previously sequenced organisms (408). For example, the control unit 104 can obtain the population samples 122 from the reference genomic data 115.

The process 400 includes determining one or more values representing the frequency of specific diplotypes occurring within the genetic sequences of previously sequenced organisms (410). For example, the custom prior model 120 of the control unit 104 can generate matrix 212 or matrix 310—e.g., as shown and described in reference to FIGS. 2 and 3.

The process 400 includes generating, using the one or more values representing the frequency of specific diplotypes occurring within the genetic sequences of previously sequenced organisms, an indication that the variant of the first joint diplotype candidate is an actual variant (412). For example, the custom prior model 120 and the variant identification engine 130 can generate an indication of one or more identified variants 136, such as a SNP variant or INDEL variant.

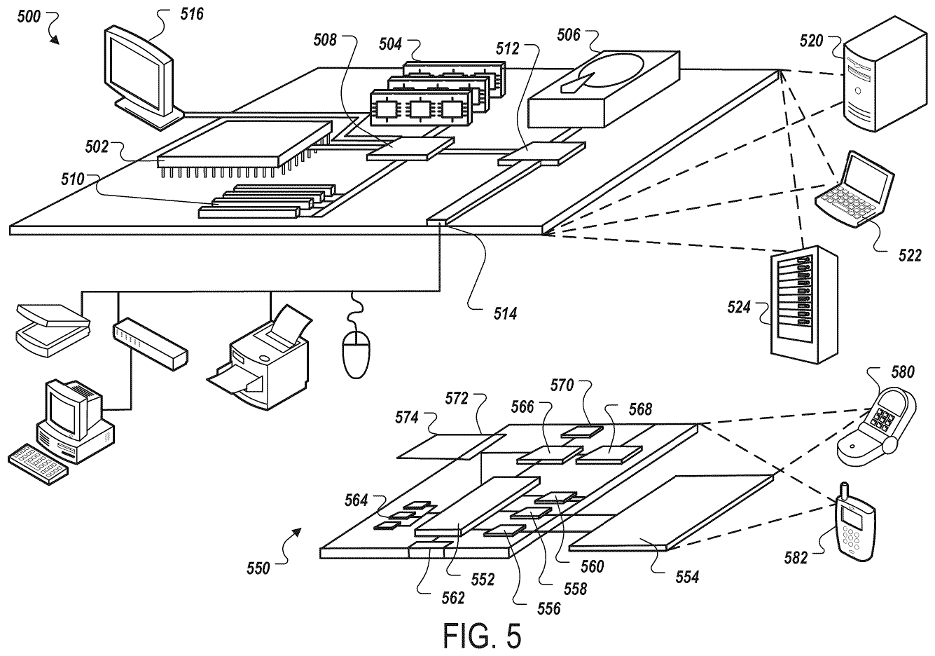

FIG. 5 is a diagram illustrating an example of a computing system used for multi-region joint detection. The computing system includes computing device 500 and a mobile computing device 550 that can be used to implement the techniques described herein. For example, one or more components of the system 100 could be an example of the computing device 500 or the mobile computing device 550, such as a computer system implementing the control unit 104 or various engines or models included therein or communicably connected to.

The computing device 500 is intended to represent various forms of digital computers, such as laptops, desktops, workstations, personal digital assistants, servers, blade servers, mainframes, and other appropriate computers. The mobile computing device 550 is intended to represent various forms of mobile devices, such as personal digital assistants, cellular telephones, smart-phones, mobile embedded radio systems, radio diagnostic computing devices, and other similar computing devices. The components shown here, their connections and relationships, and their functions, are meant to be examples only, and are not meant to be limiting.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses may be used, as appropriate, along with multiple memories and types of memory. In addition, multiple computing devices may be connected, with each device providing portions of the operations (e.g., as a server bank, a group of blade servers, or a multi-processor system). In some implementations, the processor 502 is a single threaded processor. In some implementations, the processor 502 is a multi-threaded processor. In some implementations, the processor 502 is a quantum computer.

The memory 504 stores information within the computing device 500. In some implementations, the memory 504 is a volatile memory unit or units. In some implementations, the memory 504 is a non-volatile memory unit or units. The memory 504 may also be another form of computer-readable medium, such as a magnetic or optical disk.

The storage device 506 is capable of providing mass storage for the computing device 500. In some implementations, the storage device 506 may be or include a computer-readable medium, such as a floppy disk device, a hard disk device, an optical disk device, or a tape device, a flash memory or other similar solid-state memory device, or an array of devices, including devices in a storage area network or other configurations. Instructions can be stored in an information carrier. The instructions, when executed by one or more processing devices (for example, processor 502), perform one or more methods, such as those described above. The instructions can also be stored by one or more storage devices such as computer- or machine readable mediums (for example, the memory 504, the storage device 506, or memory on the processor 502). The high-speed interface 508 manages bandwidth-intensive operations for the computing device 500, while the low-speed interface 512 manages lower bandwidth-intensive operations. Such allocation of functions is an example only. In some implementations, the high speed interface 508 is coupled to the memory 504, the display 516 (e.g., through a graphics processor or accelerator), and to the high-speed expansion ports 510, which may accept various expansion cards (not shown). In the implementation, the low-speed interface 512 is coupled to the storage device 506 and the low-speed expansion port 514. The low-speed expansion port 514, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device, such as a mobile computing device 550. Each of such devices may include one or more of the computing device 500 and the mobile computing device 550, and an entire system may be made up of multiple computing devices communicating with each other.

The mobile computing device 550 includes a processor 552, a memory 564, an input/output device such as a display 554, a communication interface 566, and a transceiver 568, among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate.

The processor 552 can execute instructions within the mobile computing device 550, including instructions stored in the memory 564. The processor 552 may be implemented as a chipset of chips that include separate and multiple analog and digital processors. The processor 552 may provide, for example, for coordination of the other components of the mobile computing device 550, such as control of user interfaces, applications run by the mobile computing device 550, and wireless communication by the mobile computing device 550.

The processor 552 may communicate with a user through a control interface 558 and a display interface 556 coupled to the display 554. The display 554 may be, for example, a TFT (Thin-Film-Transistor Liquid Crystal Display) display or an OLED (Organic Light Emitting Diode) display, or other appropriate display technology. The display interface 556 may include appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a SIMM (Single In Line Memory Module) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provide as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory and/or NVRAM memory (nonvolatile random access memory), as discussed below. In some implementations, instructions are stored in an information carrier such that the instructions, when executed by one or more processing devices (for example, processor 552), perform one or more methods, such as those described above. The instructions can also be stored by one or more storage devices, such as one or more computer- or machine-readable mediums (for example, the memory 564, the expansion memory 574, or memory on the processor 552). In some implementations, the instructions can be received in a propagated signal, for example, over the transceiver 568 or the external interface 562.

The mobile computing device 550 may communicate wirelessly through the communication interface 566, which may include digital signal processing circuitry in some cases. The communication interface 566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), LTE, 5G/6G cellular, among others. Such communication may occur, for example, through the transceiver 568 using a radio frequency. In addition, short-range communication may occur, such as using a Bluetooth, Wi-Fi, or other such transceiver (not shown). In addition, a GPS (Global Positioning System) receiver module 570 may provide additional navigation- and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, among others) and may also include sound generated by applications operating on the mobile computing device 550.

The mobile computing device 550 may be implemented in a number of different forms, as shown in the figure. For example, it may be implemented as a cellular telephone 580. It may also be implemented as part of a smart-phone 582, personal digital assistant, or other similar mobile device.

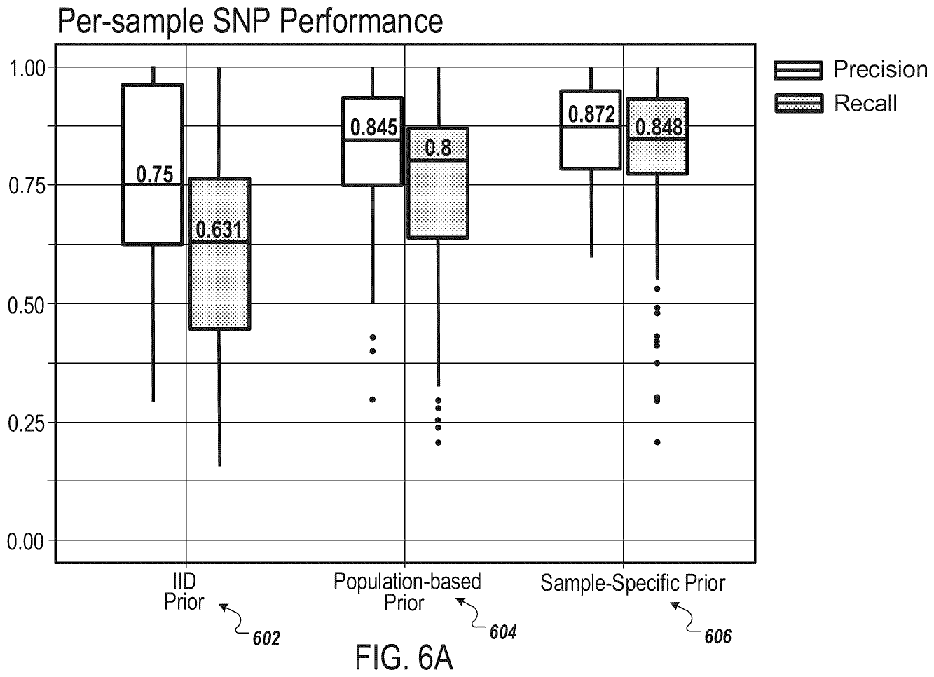

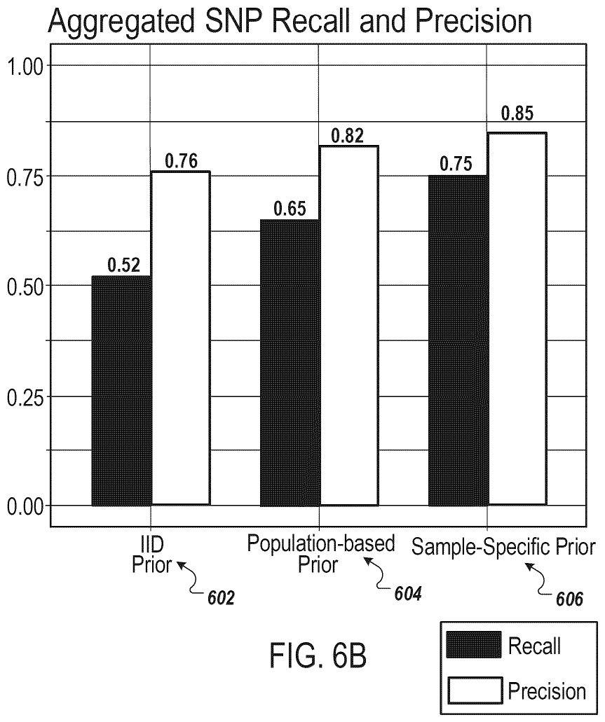

FIG. 6A and FIG. 6B are diagrams illustrating performance of the described techniques. In particular, FIG. 6A and FIG. 6B include graphs showing a benchmark of multi-region joint detection SNP calling performance using three different prior models. In reference to FIG. 1, the performance graphs of FIG. 6A and FIG. 6B show alternative implementations of the custom prior model 120. The alternative implementations include an IID prior implementation 602 (e.g., where prior probability values used to identify variants are independent and identically distributed). An example expression for determining IID prior values can include

$${( 1 - {p}_{v} )}^{N \cdot {N}_{p} - {N}_{v}} {( \frac{{p}_{v}}{9} )}^{{N}_{v}}$$

where Nᵥ represents a number of variants, Nₚ represents a number of active positions, such as 3 positions along a reference genome, and pᵥ represents a probability of a variant, such as 0.01, another static value, or a dynamic value.

The alternative implementations include a population-based implementation 604. An example of such a population-based implementation of the custom prior model 120 is described in reference to FIG. 2. The alternative implementations include a sample-specific implementation 606. An example of such a sample-specific implementation of the custom prior model 120 is described in reference to FIG. 3.

FIG. 6A and FIG. 6B show performance of the various alternative implementations detecting SNP variants per sample (e.g., in FIG. 6A) and in aggregate (e.g., in FIG. 6B). The implementations were used for detecting variants in the PMS2 high homology regions (exon12-15).

The population-based prior 604 was derived from genotypes for approximately 150 cell line samples using long-range (LR) polymerase chain reaction (PCR) next-generation sequencing (NGS) data. The sample-specific prior 606 was derived from the same LR-PCR NGS data plus a reference panel for the 1000 Genomes Project (1kGP). Both the population-based prior 604 and sample-specific prior 606 offer significant increases in precision and recall for small variant calling. The sample-specific prior 606 offers the highest performance for both SNP precision and recall.

A number of implementations have been described. Nevertheless, it will be understood that various modifications may be made without departing from the spirit and scope of the disclosure. For example, various forms of the flows shown above may be used, with steps re-ordered, added, or removed.

Multiple technological improvements and advantages of the present disclosure have been provided herein. However, the present disclosure is not limited to those improvements and advantages. Instead, a person of ordinary skill in the art would recognize many other technological improvements and advantages that result from the MRJD methods that use custom priors generated from a population based described herein, the entire list cannot be fully enumerated. Examples of these other technological improvements or advantages include accurate detection of variants relative to paralogous regions of a reference genome requiring performance of the operations described herein with reference to hundreds, thousands, millions, or tens of millions reference sequence locations. In some cases, the present disclosure enables performance of operations that could not reasonably or practicably be performed in a human mind in a reasonable amount of time given the complexity of the operations performed for set of, e.g., tens of millions of reference sequence locations.

As another example, the improvements to variant call accuracy achieved by the present disclosure reduce downstream processing that needs to be performed and improve the accuracy thereof. For example, a downstream process such as tertiary analysis performed using the variants identified by the present disclosure are more accurate and efficient, as they are operating on more accurate variant sets. Tertiary analysis on these more accurate variant sets can be executed and the results trusted in a more routine manner than conventional tertiary analysis operations that may be executed on less accurate variant sets, which can lead to analysis that needs to be performed again, results that cannot be trusted as much as those results generated by the present disclosure, or a combination of both.

Persons of ordinary skill in the art would recognize these, and other, improvements and advantages that naturally follow from implementation of the MRJD methods that use custom priors generated from a population based described herein.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, e.g., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

In certain embodiments, processing data sets as described herein can reduce the complexity and/or dimensionality of large and/or complex data sets. A non-limiting example of a complex data set includes sequence read data generated from one or more test subjects and a plurality of reference subjects of different ages and ethnic backgrounds. In some embodiments, data sets can include from thousands to millions of sequence reads for each test and/or reference subject.

Embodiments of the invention can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the invention, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Particular embodiments of the invention have been described. Other embodiments are within the scope of the following claims. For example, the steps recited in the claims can be performed in a different order and still achieve desirable results.

What is claimed is:

1. A method for identifying variants using multi-region joint detection in genetic sample sequences, the method comprising:
obtaining a plurality of haplotypes from one or more reads of a biological sample;
generating, using the plurality of haplotypes, a set of candidate diplotypes mapped to at least two different regions in a reference sequence;
generating a set of joint diplotype candidates comprising two or more candidate diplotypes of the set of candidate diplotypes, wherein the set of joint diplotype candidates includes a first joint diplotype candidate indicating a variant in at least one base from the reference sequence;
querying, for diplotypes at one or more locations of the at least two different regions in the reference sequence, a population database comprising genetic sequences of previously sequenced organisms;
determining one or more values representing the frequency of specific diplotypes occurring within the genetic sequences of previously sequenced organisms; and
generating, using the one or more values representing the frequency of specific diplotypes occurring within the genetic sequences of previously sequenced organisms, an indication that the variant of the first joint diplotype candidate is an actual variant.

2. The method of claim 1, comprising:
identifying the at least two different regions in the reference sequence as paralogous or homologous regions.

3. The method of claim 1, wherein generating the set of joint diplotype candidates comprises:
generating permutations of haplotypes, from among the plurality of haplotypes, and mapping regions, from among the at least two different regions in the reference sequence, for all haplotypes of the plurality of haplotypes and all regions of the at least two different regions in the reference sequence.

4. The method of claim 1, wherein determining the one or more values representing the frequency of specific diplotypes occurring within the genetic sequences of previously sequenced organisms comprises:
determining a quantity for each unique diplotype in the genetic sequences of previously sequenced organisms, wherein the quantity represents a number of distinct genetic sequences with the given unique diplotype at a given location; and
determining a quantity of the genetic sequences of previously sequenced organisms.

5. The method of claim 1, wherein determining the one or more values representing the frequency of specific diplotypes occurring within the genetic sequences of previously sequenced organisms comprises:
determining a quantity for each unique diplotype in the genetic sequences of previously sequenced organisms, wherein the quantity represents a number of distinct genetic sequences with the given unique diplotype at a given location;
determining a quantity of the genetic sequences of previously sequenced organisms;
determining one or more similarity values representing a similarity between a portion of the one or more reads of the biological sample and portions of the genetic sequences of previously sequenced organisms at one or more positions not included in the at least two different regions in the reference sequence; and
adjusting, using the one or more similarity values, values representing the quantity for each unique diplotype relative to the quantity of the genetic sequences; and
determining the adjusted values as the one or more values representing the frequency of specific diplotypes occurring within the genetic sequences of previously sequenced organisms.

6. The method of claim 5, wherein determining the one or more similarity values comprises:
determining a number of variants between the portion of the one or more reads of the biological sample and the portions of the genetic sequences of previously sequenced organisms at the one or more positions not included in the at least two different regions in the reference sequence.

7. The method of claim 1, wherein generating, using the one or more values representing the frequency of specific diplotypes occurring within the genetic sequences of previously sequenced organisms, an indication that the variant of the first joint diplotype candidate is an actual variant comprises:
generating, using the one or more values representing the frequency of specific diplotypes occurring within the genetic sequences of previously sequenced organisms, one or more values representing an a-priori probability;
generating, using the generated one or more values representing the a-priori probability, one or more values representing an a-posteriori probability.

8. The method of claim 7, wherein generating the one or more values representing the a-priori probability comprises:
combining, based on a location and composition of each diplotype in the first joint diplotype candidate, two or more of the one or more values representing the frequency of specific diplotypes occurring within the genetic sequences of previously sequenced organisms.

9. A system for identifying variants using multi-region joint detection in genetic sample sequences, comprising:
one or more processors; and
machine-readable media interoperably coupled with the one or more processors and storing one or more instructions that, when executed by the one or more processors, perform operations comprising:
obtaining a plurality of haplotypes from one or more reads of a biological sample;
generating, using the plurality of haplotypes, a set of candidate diplotypes mapped to at least two different regions in a reference sequence;
generating a set of joint diplotype candidates comprising two or more candidate diplotypes of the set of candidate diplotypes, wherein the set of joint diplotype candidates includes a first joint diplotype candidate indicating a variant in at least one base from the reference sequence;
querying, for diplotypes at one or more locations of the at least two different regions in the reference sequence, a population database comprising genetic sequences of previously sequenced organisms;
determining one or more values representing the frequency of specific diplotypes occurring within the genetic sequences of previously sequenced organisms; and
generating, using the one or more values representing the frequency of specific diplotypes occurring within the genetic sequences of previously sequenced organisms, an indication that the variant of the first joint diplotype candidate is an actual variant.

10. The system of claim 9, the operations further comprising:
identifying the at least two different regions in the reference sequence as paralogous or homologous regions.

11. The system of claim 9, wherein generating the set of joint diplotype candidates comprises:
generating permutations of haplotypes, from among the plurality of haplotypes, and mapping regions, from among the at least two different regions in the reference sequence, for all haplotypes of the plurality of haplotypes and all regions of the at least two different regions in the reference sequence.

12. The system of claim 9, wherein determining the one or more values representing the frequency of specific diplotypes occurring within the genetic sequences of previously sequenced organisms comprises:
determining a quantity for each unique diplotype in the genetic sequences of previously sequenced organisms, wherein the quantity represents a number of distinct genetic sequences with the given unique diplotype at a given location; and
determining a quantity of the genetic sequences of previously sequenced organisms.

13. The system of claim 9, wherein determining the one or more values representing the frequency of specific diplotypes occurring within the genetic sequences of previously sequenced organisms comprises:
determining a quantity for each unique diplotype in the genetic sequences of previously sequenced organisms, wherein the quantity represents a number of distinct genetic sequences with the given unique diplotype at a given location;
determining a quantity of the genetic sequences of previously sequenced organisms;
determining one or more similarity values representing a similarity between a portion of the one or more reads of the biological sample and portions of the genetic sequences of previously sequenced organisms at one or more positions not included in the at least two different regions in the reference sequence; and
adjusting, using the one or more similarity values, values representing the quantity for each unique diplotype relative to the quantity of the genetic sequences; and
determining the adjusted values as the one or more values representing the frequency of specific diplotypes occurring within the genetic sequences of previously sequenced organisms.

14. The system of claim 9, wherein determining the one or more similarity values comprises:
determining a number of variants between the portion of the one or more reads of the biological sample and the portions of the genetic sequences of previously sequenced organisms at the one or more positions not included in the at least two different regions in the reference sequence.

15. The system of claim 9, wherein generating, using the one or more values representing the frequency of specific diplotypes occurring within the genetic sequences of previously sequenced organisms, an indication that the variant of the first joint diplotype candidate is an actual variant comprises:
generating, using the one or more values representing the frequency of specific diplotypes occurring within the genetic sequences of previously sequenced organisms, one or more values representing an a-priori probability;
generating, using the generated one or more values representing the a-priori probability, one or more values representing an a-posteriori probability.

16. The system of claim 15, wherein generating the one or more values representing the a-priori probability comprises:
combining, based on a location and composition of each diplotype in the first joint diplotype candidate, two or more of the one or more values representing the frequency of specific diplotypes occurring within the genetic sequences of previously sequenced organisms.

17. A non-transitory computer-readable medium storing one or more instructions executable by a computer system to perform for identifying variants using multi-region joint detection in genetic sample sequences, the operations comprising:
obtaining a plurality of haplotypes from one or more reads of a biological sample;
generating, using the plurality of haplotypes, a set of candidate diplotypes mapped to at least two different regions in a reference sequence;
generating a set of joint diplotype candidates comprising two or more candidate diplotypes of the set of candidate diplotypes, wherein the set of joint diplotype candidates includes a first joint diplotype candidate indicating a variant in at least one base from the reference sequence;
querying, for diplotypes at one or more locations of the at least two different regions in the reference sequence, a population database comprising genetic sequences of previously sequenced organisms;
determining one or more values representing the frequency of specific diplotypes occurring within the genetic sequences of previously sequenced organisms; and
generating, using the one or more values representing the frequency of specific diplotypes occurring within the genetic sequences of previously sequenced organisms, an indication that the variant of the first joint diplotype candidate is an actual variant.

18. The non-transitory computer-readable medium of claim 17, wherein determining the one or more values representing the frequency of specific diplotypes occurring within the genetic sequences of previously sequenced organisms comprises:
determining a quantity for each unique diplotype in the genetic sequences of previously sequenced organisms, wherein the quantity represents a number of distinct genetic sequences with the given unique diplotype at a given location;
determining a quantity of the genetic sequences of previously sequenced organisms;
determining one or more similarity values representing a similarity between a portion of the one or more reads of the biological sample and portions of the genetic sequences of previously sequenced organisms at one or more positions not included in the at least two different regions in the reference sequence; and
adjusting, using the one or more similarity values, values representing the quantity for each unique diplotype relative to the quantity of the genetic sequences; and
determining the adjusted values as the one or more values representing the frequency of specific diplotypes occurring within the genetic sequences of previously sequenced organisms.

19. The non-transitory computer-readable medium of claim 17, wherein generating, using the one or more values representing the frequency of specific diplotypes occurring within the genetic sequences of previously sequenced organisms, an indication that the variant of the first joint diplotype candidate is an actual variant comprises:
generating, using the one or more values representing the frequency of specific diplotypes occurring within the genetic sequences of previously sequenced organisms, one or more values representing an a-priori probability;
generating, using the generated one or more values representing the a-priori probability, one or more values representing an a-posteriori probability.

20. The non-transitory computer-readable medium of claim 19, wherein generating the one or more values representing the a-priori probability comprises:
combining, based on a location and composition of each diplotype in the first joint diplotype candidate, two or more of the one or more values representing the frequency of specific diplotypes occurring within the genetic sequences of previously sequenced organisms.
